(19) United States
(12) Patent Application Publication
(10) Pub. No.: US 2026/0224668 A1
(43) Pub. Date: Aug. 6, 2026

(54) AMNIOTIC AND CHORIONIC COLLAGEN FOR OCULAR SURFACE HEALING

(71) Applicant: Cetel Scientific LLC, Rye, NY (US)

(72) Inventor: Celso Tello, Rye, NY (US)

(21) Appl. No.: 19/155,044

(22) PCT Filed: Mar. 29, 2024
(86) PCT No.: PCT/US2024/022302

§ 371 Date: Aug. 8, 2025

Related U.S. Application Data
(63) Continuation of application No. PCT/US2023/065175, filed on Mar. 30, 2023.
(60) Provisional application No. 63/362,190, filed on Mar. 30, 2022.

(51) Int. Cl. A61K 38/39; A61K 9/00; A61K 9/06; A61K 9/08; A61K 47/10; A61K 47/32; A61K 47/34; A61K 47/38

(52) U.S. Cl. CPC A61K 38/39; A61K 9/0048; A61K 9/06; A61K 9/08; A61K 47/10; A61K 47/32; A61K 47/34; A61K 47/38

(57) ABSTRACT

A composition of amniotic and/or chorionic collagen is provided in an eye drop or gel formulation for the treatment of ocular surface disorders, wherein the amniotic and/or chorionic collagen forms a film on an ocular surface for ocular surface regeneration. The composition may contain 0.01% (w/v) to 5.0% (w/v) of a blend of type III and type IV collagen derived from mammalian placenta amnion or chorion. A preferred placental source may be bovine. Other ingredients in the eye drop or gel compositions may include one or more of a demulcent selected from hyaluronic acid, glycerin, polyethylene glycol, sodium carboxymethylcellulose, and propylene glycol, gelling agents, a poloxamer or a carbomer or a combination thereof, a preservative, a demulcent, a soluble polyvinylpyrrolidone and a viscosity enhancer.

### CROSS-REFERENCE TO RELATED APPLICATION(S)

The present non-provisional application is a § 371 of International Application No. PCT/US2024/022302 filed on Mar. 29, 2024, which claims priority to International Application No. PCT/US2023/065175 filed on Mar. 30, 2023, which claims the benefit of U.S. Provisional Application Ser. No. 63/362,190, filed Mar. 30, 2022, the contents of which are incorporated herein by reference in its entirety.

### FIELD OF THE INVENTION

This invention pertains to the isolation of collagen from amniotic membrane and/or chorionic membranes and the preparation of topical ocular therapeutic products including eye drops and gels containing such collagen for the treatment of ocular injuries and diseases.

### BACKGROUND OF THE INVENTION:

Ocular surface disorders, such as dry eye syndrome, corneal erosions, epithelial defects, traumatic injury, chemical injury, and burns can cause discomfort, pain, and vision impairment. Treatment of ocular surface disorders requires medical and surgical intervention, both acutely and in the long term. Regardless of the underlying causes involved, the common goals of management include controlling inflammation and promoting ocular surface healing with maximal visual rehabilitation. Various medical therapies have been used to achieve these objectives, including several methods derived from placental materials. However, current treatments for these conditions have substantial limitations and often provide only temporary or insufficient relief.

The placenta is a temporary embryonic and fetal organ that begins developing from a blastocyst shortly after implantation. It plays critical roles in facilitating nutrient, gas, and waste exchange between the physically separate maternal and fetal circulations, and is an important endocrine organ, producing hormones that regulate both maternal and fetal physiology during pregnancy. The placenta connects to the fetus via the umbilical cord. The placenta has several extraembryonic membranes associated with the developing embryo, including the amnion and the chorion, which are the chorioamniotic membranes that make up the amniotic sac which surrounds and protects the embryo.

The term “amnion” is synonymous with “amniotic membrane” as used herein. The amnion is the innermost layer of the placenta and lines the inner surface of the amniotic cavity. The amnion is avascular, meaning it does not contain its own blood vessels, and is characterized by cuboidal and columnar epithelial layers, a thick basement membrane, and an avascular mesenchymal layer containing hyaluronic acid. The amnionic epithelium is connected to a basement membrane, which is then attached by filaments to a connective tissue layer. Thus, the amnion has mechanical integrity and can be peeled away from the chorion and other placental tissue. The extracellular matrix of the amnion includes collagen types I, III, IV, V, and VI, and also fibronectin, hyaluronan, biglycan, and decorin.[1]

The chorion is bound by the amnion and contains fetal blood vessels. The chorion is also fibrous with a collagenous basement membrane and reticular layer and can be separated from the amnion and other placental tissue. The extracellular matrix of the chorion has similar chemical composition to the amnion. As used herein, “placental extracts” refer to materials derived from the chorion of a placenta.

Amniotic and placental extracts, referred to herein as “A/PE”, have been shown to have wound healing qualities.[2],[3] Chorionic tissue includes trophoblast cells which form in humans within days of fertilization of the egg and provide nutrients to the embryo.[4] Trophoblast cells are protected from the maternal immune system, due to reduced expression of the major histocompatibility complex (MHC), apoptosis-inducing mechanisms, and the influence of hormones and growth factors on the cells of the immune system.[3] AP/E have been shown to have wound healing properties, associated with an increase of transforming growth factor-β (TGF-β) in the early phase of regeneration and vascular endothelial growth factor (VEGF) in the late phase, as well as with the presence of fibroblast growth factor (FGF), amplification of angiogenesis, and the increase of expression of CD31.

Collagen is a major component of both the amnion and chorion [5] [6] and collagen is believed to exert the healing effect of A/PE therapies.[7] “Collagen” is a super family of proteins having at least one triple-helical domain. At least 28 collagen variations are known.[5] [8 ] Collagen types III and IV, which are major components of amniotic and chorionic membranes, are of particular interest in this invention. Collagen type III has been shown to have wound healing properties.[9] Type IV collagen is the most abundant constituent of basement membranes that forms a network-like structure in the extracellular matrix. Type IV collagen provides a scaffold in the basement membrane with other macromolecules, such as laminins, heparan sulfate proteoglycans, fibronectin, entactin, and regulates cell adhesion and migration which are important for wound healing.[10],[11] Collagen is used in regenerative medicine, the branch of medicine that develops methods to regrow, repair or replace damaged or diseased cells, organs or tissues.[12]

Amniotic membrane and amniotic membrane extracts are known to reduce inflammation, fibrovascular ingrowth, and to facilitate epithelialization in animal and human models. Amniotic membrane is believed to play a role in the scarless wound healing process. [13],[14] An approach to therapeutic uses of placental material is amniotic membrane (AM) graft and amniotic membrane transplantation (AMT). AM graft and AMT have been used in ophthalmology for several indications because of its beneficial effects.[15] Amniotic membrane is usually obtained from amniotic tissue derived from placentas in mammals, such as humans, pigs, bovines, or horses.

Previous studies revealed that early intervention with AMT in ophthalmic surgery results in marked reduction of inflammation, rapid restoration of the ocular surface, and improved visual acuities while preventing cicatricial complications. [15] However, surgically performed AMT is expensive and may cause unnecessary surgical trauma in already compromised eyes. Furthermore, the membrane patch usually dissolves within several days so that multiple sessions of AMT may be required. Previously, a self-retaining AM mounted on a double ring system has been used to promote healing and reduce corneal scarring in a variety of ocular surface disorders, however, patients experienced ocular discomfort from the ring and incomplete healing has been reported. [17]

Previous studies have also shown that topical amniotic membrane extract (AME) has comparable effect to AMT in promoting epithelialization, decreasing inflammation, and suppressing corneal neovascularization. [18] However AME lacks the physical characteristics of a bandage and as such it cannot be used as a patch graft. [19] Another previous approach to the use of amniotic proteins to treat ocular disease and injury is the application of amniotic fluid topically delivered to the eye. [7]

An approach to the use of collagen extracts for ocular healing was disclosed in US 2008/0181967 A1 [20] involving the use of placental or umbilical extracts. US 2008/0181967 discloses a number of treatment modalities, but umbilical cord extract is predominantly type I and II collagen.

A related technology is corneal shields, which have been made from collagen. [21] Collagen corneal shields are a bandage lens device for ocular surface protection following surgery and in traumatic and nontraumatic corneal conditions. They are available with dissolution times up to 72 hours. [cite to Willoughby] The use of collagen corneal shields for drug delivery has been disclosed, by soaking the devices in a drug solution prior to application. [21]

A related technology is the PROKERA® SLIM, which is a self-retaining biologic corneal bandage made with cryopreserved amniotic membrane grafts fastened to an ophthalmic conformer. This product is aseptically processed from donated human placenta tissue. The PROKERA® SLIM is indicated for use in eyes in which ocular surface cells are damaged or underlying the stroma is inflamed or scarred. The PROKERA® SLIM includes a plastic ring that is not resorbable and must be removed. The PROKERA® SLIM can remain inserted in an eye for up to 29 days. Thus, as compared to the invention described herein, the PROKERA® SLIM is not fully resorbable and has non-resorbable parts that must be removed. A further shortcoming is that the amniotic membrane itself is not in the shape of a contact lens. Thus, the contact with the cornea is less than optimum. Moreover, the ring may cause discomfort.

There is a need for new, more effective treatments that promote ocular surface regeneration. U.S. Pat. No. 9,295,753 B1 disclosed a method of producing a complex containing human or mammalian A/PE for ophthalmic use along with a method of controlling the degradation over time according to the intended use. US patent publication US 20160220485 disclosed a method for developing an A/PE lens-shaped patch to cover and protect the cornea in various ocular surface disorders.

There remains a need for effective, convenient application of A/PE collagen types III and IV to heal surface injuries to the cornea as is provided by the instant invention.

### SUMMARY OF THE INVENTION

This invention provides type III and IV collagen derived from mammalian amnion and chorion for ocular surface regeneration. The collagen of this method can be formed into eye drops and gels. It may be useful to boost or restore ocular surface health and maintain/facilitate clear vision. In an embodiment, bovine placentas are preferred.

The amniotic and placental collagen as disclosed herein is mostly types III and IV collagen, which has been shown to promote tissue repair and regeneration. The amniotic and placental collagen is isolated and purified from the amniotic membrane using standard techniques, ensuring that it is potent and free from contaminants and pathogens.

Collagen is resorbable under physiological conditions including when in contact with a cornea. The collagen used herein may be crosslinked to increase the degradation time of the inventive compositions. For example, the collagen may have 0.05% to 0.75% (w/w) riboflavin incorporated into it, and the composition may be exposed to UV radiation to crosslink the collagen to adjust the degradation time.

In an embodiment, this invention provides a composition of amniotic and/or chorionic collagen in an eye drop or gel formulation for the treatment of ocular surface disorders, including 0.01% (w/v) to 5.0% (w/v) of a blend of type III and type IV collagen derived from mammalian amnion or chorion, and a demulcent selected from hyaluronic acid, glycerin, polyethylene glycol, sodium carboxymethylcellulose, and propylene glycol, in an isotonic solution, wherein the amniotic and/or chorionic collagen forms a film on the surface of the cornea ocular surface regeneration.

The composition may further include a gelling agent selected from hydroxypropyl methylcellulose, hydroxypropyl cellulose, and methyl cellulose present in 1.0% to 15% (w/v), and a poloxamer or carbomer.

The composition may be an eyedrop formulation or a gel as a viscous transparent liquid applied directly to the cornea of an eye.

In an embodiment, the amniotic or chorionic collagen is obtained from a bovine placenta. In an embodiment the collagen is obtained from a placental amnion wherein the chorion is removed.

In an embodiment, the blend of type III and type IV collagen is crosslinked to control its degradation rate.

In an embodiment, the blend of type III and type IV collagen is dispersed in a water-based solvent at room temperature to reach a concentration of 0.01% to 5.0% wt %, and one or more excipients selected from a gelling agent, a demulcent, and a viscosity enhancer is added to form a gel.

In an embodiment, the blend of type III and type IV collagen in an isotonic solution is heated together at 37° C. with one or more excipients selected from a gelling agent, a demulcent, and a viscosity enhancer, to form a gel upon cooling.

In an embodiment, the blend of type III and type IV collagen is blended with a thermoresponsive polymer at room temperature in an isotonic solution, to form a solution that forms a gel on application to the relatively elevated temperature of the ocular surface. A thermoresponsive polymer may be a poloxamer.

In an embodiment, a method is provided for isolating and purifying a substantially cell-free amniotic or chorionic mixture of primarily type III and type IV collagen from a mammalian placenta. The method includes separating an amnion and/or chorion from the placenta and soaking the amnion and/or chorion tissue in dilute acid such as 0.5M acetic acid for 7-14 days at about 4° C., with vigorous agitation daily, decanting the supernatant and filtering to removing solid material, and concentrating the supernatant to provide a solution containing a mixture of primarily type III and type IV collagen.

In an embodiment, a blend of primarily type III and type IV collagen is obtained from amnion and/or chorion from a placenta, by incubating the amnion and/or chorion tissue in 0.5% pepsin for 24 to 72 hours at 37° C. and decanting the supernatant and filtering to remove solid material. The supernatant is adjusted to pH 7.0 to 7.4 with sodium hydroxide solution and concentrated using ultrafiltration. The remaining solution is sterilized by filtration or gamma irradiation to provide a solution containing a mixture of primarily type III and type IV collagen.

In an embodiment, inventive compositions treat a medical condition selected from dry eye syndrome, corneal trauma, corneal inflammation, corneal burn injury, or corneal ulcers.

### DETAILED DESCRIPTION OF THE INVENTION

This invention provides topical ocular formulations, for example as eye drops or gels, containing substantially cell-free collagen of primarily type III and IV collagen as an active agent with healing properties for injuries and diseases of the ocular surface. The collagen is obtained from mammalian amniotic or chorionic membranes or both. The eye-drops or ocular gel may be applied directly to the surface of an eye for ocular surface regeneration, and to treat ocular surface disorders. The formulations may form a film on the surface of the eye with therapeutic properties. By the use of crosslinked collagen, the lifetime of the gel before the collagen is resorbed and dissolves can be extended from several minutes to several hours.

As used herein, the word “about” means +/−20% of a stated value. The term “w/v” means mass concentration of weight to volume. The term “w/v” is synonymous with “wt %.” Type III and IV collagen is also termed “type III/IV” collagen.

### Preparation of Amniotic and Chorionic Collagen

The collagen material of this invention is obtained from amniotic membranes and/or chorionic membranes from the placentas of mammals. The placentas may be obtained from any mammalian species such as bovine, human, porcine, equine, or others. Bovine placental materials may be preferred because cattle are very large animals with large placentas that are easier to work with and have high yields of collagen. Also, there may be special herds of cattle (closed herds) kept for sourcing bovine-derived medical products, and placental materials from multiple animals may be combined from properly sourced animals. By contrast, FDA regulations may prevent combining human placentas.

The U.S. Food and Drug Administration (FDA) has established a guidance for new products containing biological materials sourced from cattle. Europe and parts of Asia will not accept materials made from European sources and only limited sources from USA. Bovine spongiform encephalopathy (BSE, Mad Cow Disease) has had a major impact across the globe and regulatory bodies are increasingly cautious about products sourced from bovines.

Closed herds in New Zealand and the US have strict guidelines that must be met to qualify as a closed herd. These restrictions include documentation of the lineage of each animal, no commingling with other herds, increased US Department of Agriculture (USDA), or New Zealand Ministry for Primary Industries (MPI) scrutiny, increased veterinarian scrutiny, and animal husbandry controls for ranch practices, breeding, and herd health. The New Zealand MPI is responsible for food safety in New Zealand. The animals have never been fed ruminant or other animal derived protein. No steroids or growth hormones are used. Calves are not given antibiotics. The herd must also be certified to have never had a BSE case. All harvesting is conducted under the surveillance of inspectors (USDA or MPI). Materials are harvested in an abattoir (slaughterhouse) specifically built for the closed herd to control all aspects of the process. Controls are also in place during harvesting to minimize or eliminate contact with brain and spinal cord tissues believed to transmit BSE.

The abattoir operates as a cGMP facility with all process and procedures written and approved. This ensures reproducibility of the products.

New Zealand sourced materials provide a layer of protection from potential diseases. New Zealand has never had a BSE case or many of the other bovine diseases that are found in other parts of the world (USA included). Closed herds in New Zealand test for diseases as required by the FDA. The New Zealand MPI has a higher level of requirements for animal husbandry, with most (>90%) cattle being organically raised. A closed herd, for example, is kept at Huruiki Farms Ltd., Hikurangi, New Zealand.

Placentas used in this invention should be obtained as soon as possible after a birth but can be stored for up to three days without degradation of the collagenous material. If the placenta is human, the mother should be screened for HIV, HBV, HCV, HTLV, syphilis, CMV, and other pathogens known to contaminate placental tissue. This step may not be necessary in placentas obtained from cattle in restricted herds, but some diseases may be present that should be screened, such as bovine tuberculosis, and mad cow disease.

An exemplary method of processing placental tissue comprises separating the umbilical cord from the placental disc and then separating the chorioamniotic membrane from the bulk of the placenta. Optionally, the amniotic membrane is separated from the chorionic membrane. The placental tissue, i.e., the chorioamniotic membrane tissue, may optionally be stored in a sterile, preferably buffered, saline solution, such as 0.9% sterile NaCl solution, preferably with refrigeration at a temperature of at least 4° C. in preparation for making a solution or gel of this invention.

In an embodiment, the use of collagen derived from amniotic membrane, without chorionic membrane, is preferred because the chorion is vascularized, and even though it may be exsanguinated, i.e., drained of blood, it may be difficult to completely remove blood from the placental material. The residual blood may cause a brown discoloration in the inventive compositions. By contrast, the amnion has no vascularization, so removal of blood stain should be easier. Collagen obtained from amniotic membrane tissue is therefore cleaner and has less extraneous material that could discolor the final products. Colorless compositions are desirable to minimize visual disruption for the patient in the inventive products.

Most prior art sources of amniotic or placental extracts involve physical disruptions such as chopping, pulverizing, or homogenizing tissue in blender and separating the supernatant containing collagen from solids by e.g., centrifugation or filtration to remove solids. [22] However, the resulting materials are fairly crude and not cell free. Others have used methods such as incubating chorioamniotic tissue with any of various salts, termed “osmotic shock”, or with a detergent, either of which is said to cause cells to burst. [6]

The instant invention employs mild chemical methods that extract collagen from chorioamniotic tissue to provide collagen high in types III and IV collagen having substantial healing qualities. Two exemplary methods are shown below.

### Method 1: Acid Extraction

Fresh bovine placentas are dissected to separate the amniotic membrane and/or chorion membrane or both. The amnion and/or chorion tissue may be rinsed thoroughly with distilled water to remove any residual blood or tissue. The material may optionally be frozen overnight and chopped or shredded.

The tissue is placed in a container and covered with 0.5 M acetic acid solution. The tissue should be completely submerged in the solution.

Allow the tissue to soak in the acid solution for 7-14 days at reduced temperature, such as 4° C., with vigorous agitation daily.

After the soaking period, the supernatant is decanted away from the solids to remove the tissue from the acid solution and discard it.

Filter the supernatant through a cheesecloth or filter paper to remove any residual tissue fragments.

Adjust the pH of the solution to 7.0-7.4 using 2 M sodium hydroxide.

Concentrate the collagen solution using an ultrafiltration system.

Sterilize the collagen solution by filtration or gamma irradiation.

The primarily type III and type IV collagen obtained thereby may optionally be lyophilized to remove water.

### Method 2: Pepsin Extraction

Fresh bovine placentas are dissected to separate the amniotic membrane and/or chorion membrane or both. The amnion and/or chorion tissue may be rinsed thoroughly with distilled water to remove any residual blood or tissue. The material may optionally be frozen overnight and chopped or shredded.

Place the tissue in a container and cover with 0.5% pepsin solution. The tissue should be completely submerged in the solution.

Incubate the tissue in the pepsin solution for 24-72 hours at 37° C., with occasional stirring.

After the incubation period, remove the tissue from the pepsin solution and discard it.

Adjust the pH of the remaining solution to 7.0-7.4 using 2 M sodium hydroxide.

Concentrate the collagen solution using an ultrafiltration system.

Sterilize the collagen solution by filtration or gamma irradiation, and optionally lyophilize to provide dry collagen.

Both methods can yield high-quality collagen suitable for use in various biomedical applications, such as tissue engineering, regenerative medicine, wound healing, and drug delivery. The collagen obtained from either of these methods is primarily type III and IV collagen.

It is important to follow strict protocols for tissue collection, handling, and processing to ensure the safety and efficacy of the final product. In addition, different types of collagens have different degradation rates. This property allows the manipulation of the absorption time of collagen in different embodiments of the invention. Absorption rates may also be manipulated by the nature of crosslinking with chemical and/or thermal bonding.

### Compositions

In an embodiment, purified amniotic and/or chorionic collagen is an active ingredient that can be formulated into various types of compositions and delivery systems, including eye drops and gels for easy and convenient administration. The amniotic and/or chorionic collagen may be a blend of type III and type IV collagen. The compositions may include eyedrops having 0.01% to 5.0% collagen (w/v) in an isotonic aqueous solution. In another embodiment, the inventive compositions may have 0.01% to 5.0% collagen (w/w) in a gel, ointment, or paste, that is, a viscous transparent liquid applied directly to the surface of an eye. Products with lower concentrations, such as 0.01% to 0.50% collagen, may be useful as over the counter medications, not requiring a prescription. Higher concentrations, such as 0.25% to 5.0% may be prescription only.

Typical excipients that may be useful in this invention include gelling agents, a poloxamer or a carbomer or a combination thereof, a preservative, a demulcent, and a viscosity enhancer. A soluble polyvinylpyrrolidone (also called povidone), such as Kollidon® 12 PF, 17 PF, 30, or 90 may be used. Povidone may act as an ocular lubricant and emulsifier. In an embodiment, the composition is preservative-free.

Gelling agents include a cellulose derivative such as hydroxypropyl methylcellulose, hydroxypropyl cellulose, and methyl cellulose. These agents may be used in 1.0% to 15% (w/v) of the final composition.

Poloxamers are a class of water-soluble nonionic triblock copolymers formed by polar (polyethylene oxide) and non-polar (polypropylene oxide) blocks which confer amphiphilic and surface active properties to the polymers. Poloxamers are also known by the trade names Pluronic®, Kolliphor®, and Synperonic™.

Carbomers are poly(acrylic acid) (PAA) and with the formula (CH2-CHCO2H)ₙ, i.e., having a carbon backbone with alternating carboxyl groups. For example, Carbopol® 940 is a poloxamer used in many pharmaceutical products. Carbopol® 940 is a rheology modifier capable of providing high viscosity and forms sparkling clear gels or hydro-alcoholic gels and creams. A typical concentration is 0.2 to 1.0% (w/v). Carbomers are widely used in pharmaceuticals and cosmetics and can act as film forming agents that can help disperse the placental collagen of this invention as a film on the surface of an eye. A typical concentration is 0.2 to 1.0 wt % (w/v).

Demulcents, that is, a mucilaginous or oleaginous preparation that forms a soothing protective film over a mucus membrane, may be added to the inventive compositions. In an embodiment, several demulcents may be added to collagen to enhance its topical ophthalmic use, including:

Hyaluronic acid: This is a natural component of the eye that helps to lubricate and protect the surface of the eye. It can be added to collagen to enhance its lubricating and hydrating properties.

Glycerin: This is a humectant that helps to retain moisture in the eyes. It can be added to collagen to improve its moisturizing properties.

Polyethylene glycol: This is a water-soluble polymer that is often used as a lubricant and moisturizer in ophthalmic applications. It can be added to collagen to enhance its lubricating and hydrating properties.

Sodium carboxymethylcellulose: This is a water-soluble polymer that is commonly used as a lubricant and viscosity-enhancing agent in ophthalmic formulations. It can be added to collagen to improve its lubricating and stabilizing properties.

Propylene glycol: This is a humectant and solvent that is often used in ophthalmic formulations to improve the solubility and stability of active ingredients. It can be added to collagen to improve its moisturizing and solubilizing properties.

The choice of demulcent will depend on several factors, including the specific application and the desired properties of the final product.

There are several methods exemplified below for preparing the purified collagen as a gel for topical ophthalmic use.

Cold method: In this method, collagen is dispersed in a water-based solvent at room temperature to reach a concentration of 0.01% to 5.0%. Other gelling agents such as a cellulose derivative (e.g., hydroxypropyl methylcellulose), a poloxamer or a carbomer or a combination thereof, can be added as needed. Other excipients such as preservatives, demulcents, and viscosity enhancers may be added while stirring. A soluble polyvinylpyrrolidone (also called povidone), such as Kollidon® 12 PF, 17 PF, 30, or 90 may be used. The mixture is then homogenized and allowed to hydrate, forming a gel.

Hot method: In this method, the ingredients are heated together at 37° C. to facilitate mixing and solubility. A gelling agent is added to a heated isotonic solution, followed by active ingredients and other excipients. The mixture is then stirred until all components are fully dissolved and uniform. The mixture is then allowed to cool, forming a gel.

In situ gelation method: In this method, the gel is formed in situ (i.e. at the site of application) through a chemical or physical reaction. For example, a thermoresponsive polymer such as poloxamer can be dissolved in an aqueous solution at room temperature. Upon application to the eye, the solution undergoes a phase transition to form a gel in response to the elevated temperature of the surface of an eye.

Eyedrops may be formed in a similar fashion to the gels as described above, but in a much less viscous free-flowing liquid for use as an eye drop formulation that can be applied as drops directly to an eye.

### Controlled Biodegradability

The collagen compositions of this invention degrade under physiological conditions and may be rapidly resorbed when applied to an eye. In an embodiment, it may be desirable to crosslink the collagen to increase its degradation time.

Accordingly, the compositions provided herein may be designed to degrade at a regulated rate in a specified timeframe. That is, different compositions may be specifically calibrated to degrade at pre-determined times, ranging from 15 minutes to 8 hours. A specific degradation time may be selected using chemical curing processes, such as crosslinking by gamma or e-beam sterilization, or adding a chemical crosslinker.

Many biomedical polymers are sterilized with gamma radiation or e-beam sterilization techniques. In addition to preventing future microbial or viral growth, this sterilization has the effect of partially crosslinking collagen. This method may be used on the collagen of this disclosure to decrease the rate of degradation. That is, crosslinking by gamma or e-beam will make the collagen more resistant to degradation and, as such, it may extend the degradation time by up to 2-3 times, depending on the total radiation level imposed by the gamma radiation or e-beam exposure. However, the dosage of gamma radiation or e-beam used for this purpose must be adjusted to maintain the biological activity of the active ingredients.

Alternatively, a chemical crosslinker could be used in a similar fashion. There are many biologically compatible, bi-and tri-functional chemical crosslinkers that are commercially available. These may include organic molecules terminated with azides, amines, bromides, maleimides, isocyanates, sulfides, and esters. Riboflavin can be incorporated into the inventive compositions to enhance crosslinking after exposure to UV radiation for several seconds to one hour to adjust the degradation time of the inventive compositions. Other crosslinking agents known to those of skill in the art include glutaraldehyde, 1,4-butanediol diglycidyl ether, and genipin.[6] Genipin is a nontoxic, naturally occurring crosslinking agent. It can be obtained from its parent compound, geniposide, which may be isolated from the fruits of Gardenia jasminoides. Genipin may be obtained commercially from Challenge Bioproducts Co., Ltd., 7 Alley 25, Lane 63, TzuChiang St. 404 Taichung Taiwan R.O.C. The use of genipin as a crosslinking reagent is described in U.S. Patent Application Publication No. 20030049301.

The rate of degradability of collagen matrices can be controlled by the degree of crosslinking. The more highly crosslinked the material, the slower the degradation. The crosslinking can be a factor of the collagen itself, depending on the tissue source and chemical features of the extraction, or it can be adjusted using an additive such as riboflavin and then UV irradiation, which crosslinks the collagen stands and retards the biodegradation. The degradation time can be adjusted to as long as 48 hours. The amount of riboflavin can be 0.05% to 0.75% (w/w of the entire composition). The amount of riboflavin can be about 0.3%.

In another embodiment, thermal bonding may be useful to partially denature collagen fibrils which can retard biodegradability. [23] Heating an aqueous solution of collagen at 60° C. to 80° C. for one to 20 minutes may cause partial denaturation of collagen. Partial denaturation is observed in as little as one minute at 80° C. Complete denaturation occurs after 90 minutes at 80° C. Thus, by modulating the temperature and time of heating, partial denaturation can occur.

### Therapeutic Utility

The type III/IV collagen in the compositions of this invention reduce inflammation, reduce fibrovascular ingrowth, and facilitate epithelialization. These activities will have a healing effect for diseases and injuries to the ocular surface, which include the cornea limbus, and conjunctiva. This invention provides a method of treating an ocular condition, including providing a composition comprising an amniotic and/or placental collagen, where the composition is formulated into eyedrops or a gel that can be applied topically to the surface of an injured or diseased eye. In an embodiment, the inventive compositions form a biodegradable film to cover the whole ocular surface, not limited to the cornea.

The inventive composition may be used to therapeutically treat damaged or diseased cornea, limbus and surrounding conjunctiva, in addition to other wounds and conditions. For example, the composition may be used to promote healing after injury due to inflammation, infection, trauma, surgery, and dry eye or other ocular conditions. Any treatment and/or benefit imparted to a patient's cornea, limbus and/or surrounding conjunctiva is alternatively termed “treatment of an ocular surface condition” herein.

The inventive compositions may be applied to a patient's eye to treat ocular surface disorders associated with dry eye condition. With the composition applied to an eye, it will provide protection and enhance healing of the ocular surface. The composition will dissolve over several hours and liquefy to provide additional lubrication to the ocular surface. The use of the composition may be repeated as needed to protect and maintain ocular surface health. This will provide a sustained level of treatment and may positively impact the quality of life.

In an embodiment, the inventive compositions have veterinary applications. The inventive compositions can be useful in pets, for example dogs or cats, livestock and farm animals, such as horses, bovines, goats, sheep, swine, or camelids that have suffered corneal injuries.

### Treatment of Ocular Surface Trauma Caused by Chemical or Thermal Injury (Burns)

The inventive compositions are expected to enhance healing of a damaged ocular surface caused by trauma, chemical injury, or thermal injury. After copious irrigation of the injured surface and removal of any residual chemical particles, the inventive type III and IV collagen compositions may be applied to the ocular surface as an eyedrop or a gel. Other conventional treatments can be applied at the same time. Depending on the nature of the injury and the biodegradability of the inventive composition, gel, paste, or eyedrops may be applied as frequently as hourly, or 4 times per day, then reduced to once per day or every other day.

### Post-Refractive Treatment

Postoperative complications after refractive surgery include pain, epithelial defect, and/or haze. The inventive compositions may present an effective solution to solve these critical problems when inserted post photorefractive keratectomy (PRK).

The inventive method further includes the steps of determining a timeframe required for proper healing and/or treatment of an ocular condition (“heal rate”) and selecting a degradation timeframe (degradation rate) that is the same as or greater than the expected heal rate.

### EXAMPLES

### Example 1, Eyedrop Composition

An eye drop formulation may comprise the following materials:
Type III/IV collagen as described above (pure) 10.00 mg/mL
Glycerin 5.00%
Polyethylene glycol 400 2.00%

In an isotonic solution at a pH of 6.7 (6.5 to 7.0) and an osmolality of approximately 270 mOsmol/kg, administered as eye drops.

### Example 2, Gel Composition

Type III/IV collagen as described above (pure) 25 mg/mL (2.5% w/v)
Sodium carboxymethylcellulose 1.00% (10 mg/mL)
Calcium chloride
Potassium chloride
Sodium chloride
Sodium lactate

In a buffered isotonic solution at a pH of 6.7 (6.5 to 7.0) and an osmolality of approximately 270 mOsmol/kg

The product is a clear gel packaged in low-density polyethylene single dose vials.

### Example 3, in Situ Gel Composition

Type III/IV collagen as described above (pure) 25 mg/mL (2.5% w/v)
Sodium carboxymethylcellulose 1.00% (10 mg/mL)
Polypropylene glycol 2.00%
Carbopol® 940 1.0% (carbomer)
In a buffered isotonic solution at a pH of 6.7 (6.5 to 7.0) and an osmolality of approximately 270 mOsmol/kg

The product is a free-flowing liquid for use as an eye drop. The product should be stored at <15° C. prior to use. Upon application to the eye, a gel may be formed by the carbomer from a phase transition in response to the higher temperature of the cornea of an eye.

### REFERENCES

(1) Strauss, J. F. Extracellular Matrix Dynamics and Fetal Membrane Rupture. Reproductive Sciences. February 2013, pp 140-153. https://doi.org/10.1177/1933719111424454.
(2) Hong, J. W.; Lee, W. J.; Hahn, S. B.; Kim, B. J.; Lew, D. H. The Effect of Human Placenta Extract in Wound Healing Model. Ann Plast Surg 2010, 65 (1), 96-100. https://doi.org/10.1097/SAP.0b013e3181b0bb67.
(3) Pogozhykh, 0.; Prokopyuk, V.; Figueiredo, C.; Pogozhykh, D. Placenta and Placental Derivatives in Regenerative Therapies: Experimental Studies, History, and Prospects. Stem Cells Int 2018, 2018. https://doi.org/10.1155/2018/4837930.
(4) Wang Y; Zhao S. Chapter 4, Cell Types of the Placenta. In Vascular Biology of the Placenta; Morgan & Claypool Life Sciences, 2010.
(5) Karami, A.; Tebyanian, H.; Sayyad Soufdoost, R., Motavallian, E.; Barkhordari, A.; Nourani, M. R. Extraction and Characterization of Collagen with Cost-Effective Method from Human Placenta for Biomedical Applications. World J Plast Surg 2019, 8 (3), 352-358. https://doi.org/10.29252/wjps.8.3.352.
(6) Bhatia, M. Human Placental Collagen Compositions, and Methods of Making and Using the Same. U.S. Pat. No. 8,877,180B2, November 4, 2014.
(7) Behrens, A. Use of Amniotic Fluid (Af) in Treating Ocular Disease and Injury. US20080286378A1, 2008.
(8) Ricard-Blum, S. The Collagen Family. Cold Spring Harb Perspect Biol 2011, 3 (1), 1-19. https://doi.org/10.1101/cshperspect.a004978.
(9) Kuivaniemi, H.; Tromp, G. Type III Collagen (COL3A1): Gene and Protein Structure, Tissue Distribution, and Associated Diseases. Gene. Elsevier B.V. July 30, 2019, pp 151-171. https://doi.org/10.1016/j.gene.2019.05.003.
(10) Sudhakar, A.; Kalluri, R. Molecular Mechanisms of Angiostasis. Encyclopedia of the Eye 2010, 52-59. https://doi.org/10.1016/B978-0-12-374203-2.00128-7.
(11) Sand, J. M. B.; Genovese, F.; Gudmann, N. S.; Karsdal, M. A. Type IV Collagen. Biochemistry of Collagens, Laminins and Elastin: Structure, Function and Biomarkers 2019, 37-49. https://doi.org/10.1016/B978-0-12-817068-7.00004-5.
(12) Pawelec, K. M.; Best, S. M.; Cameron, R. E. Collagen: A Network for Regenerative Medicine. J Mater Chem B 2016, 4 (40 ), 6484-6496. https://doi.org/10.1039/c6tb00807k.
(13) Tseng, S. C. G., et al. Amniotic Membrane Covering for a Tissue Surface and Devices Facilitating Fastening of Membranes. U.S. Pat. No. 7,494,802B2, 2009.
(14) Tseng, S. C. G.; Li, D. Q.; Ma, X. Suppression of Transforming Growth Factor-Beta Isoforms, TGF-13 Receptor Type II, and Myofibroblast Differentiation in Cultured Human Corneal and Limbal Fibroblasts by Amniotic Membrane Matrix. J Cell Physiol 1999, 179 (3), 325-335. https:/doi.org/10.1002/(SICI)1097-4652(199906)179:3<325::AID-JCP10>3.0.CO.2-X.
(15) Dua, H. S.; Gomes, J. A. P.; King, A. J.; Maharajan, V. S. The Amniotic Membrane in Ophthalmology. Sury Ophthalmol 2004, 49 (1), 51-77. https://doi.org/10.1016/j.survophthal.2003.10.004.
(16) Pachigolla, G.; Prasher, P.; di Pascuale, M. A.; McCulley, J. P.; McHenry, J. G.; Mootha, V. V. Evaluation of the Role of Prokera in the Management of Ocular Surface and Orbital Disorders. Eye Contact Lens 2009, 35 (4), 172-175. https://doi.org/10.1097/ICL.0b013e3181a66a12.
(17) Suri, K.; Kosker, M.; Raber, I. M.; Hammersmith, K. M.; Nagra, P. K., Ayres, B. D.; Halfpenny, C. P.; Rapuano, C. J. Sutureless Amniotic Membrane ProKera for Ocular Surface Disorders: Short-Term Results. Eye Contact Lens 2013, 39 (5), 341-347. https://doi.org/10.1097/ICL.0B013E3182A2FSFA.
(18) Liang, L.; Li, W.; Ling S; Sheha H; Li, Q. W.; Liu Z. Amniotic Membrane Extraction Solution for Ocular Chemical Burns. Clin Experiment Ophthalmol 2009, 37 (9), 855-863. https://doi.org/10.1111/j.14429071.2009.02159.x.
(19) Sheha, H.; Liang, P. L.; Hashem, H.; Ramzy, M.; Zaki, A. Amniotic Membrane Extract for Acute Ocular Chemical Burns Background: Ocular Chemical Burn Induces Devastating and Perma. Tech. Ophthal. 2010, 8 (4), 146-150.
(20) Liu, Q.; Ray, C. PLACENTAL OR UMBILICAL CORD TISSUE COMPOSITIONS. US 2008/0181967 A1, 2008.
(21) Willoughby, C. E.; Batterbury, M.; Kaye, S. B. Collagen Corneal Shields. Ther. Rev. Survey of Ophthamology 2002, 47 (2). https://doi.org/https://doi.org/10.1016/S0039-6257(01)00304-6.
(22) Murri, M. S.; Moshirfar, M.; Birdsong, O. C.; Ronquillo, Y. C.; Ding, Y.; Hoopes, P. C. Amniotic Membrane Extract and Eye Drops: A Review of Literature and Clinical Application. Clinical Ophthalmology. Dove Medical Press Ltd June 18, 2018, pp 1105-1112. https://doi.org/10.2147/OPTH.S165553.
(23) Suwa, Y.; Nam, K.; Ozeki, K.; Kimura, T.; Kishida, A.; Masuzawa, T. Thermal Denaturation Behavior of Collagen Fibrils in Wet and Dry Environment. J Biomed Mater Res B Appl Biomater 2016, 104 (3), 538-545. https://doi.org/https://doi.org/10.1002/jbm.b.33418.

1. A composition of amniotic and/or chorionic collagen in an eye drop or gel formulation for the treatment of ocular surface disorders, comprising 0.01% (w/v) to 5.0% (w/v) of a blend of type III and type IV collagen derived from mammalian placenta amnion or chorion, and a demulcent selected from the group consisting of hyaluronic acid, glycerin, polyethylene glycol, sodium carboxymethylcellulose, and propylene glycol, in an isotonic solution, wherein the amniotic and/or chorionic collagen forms a film on the surface of the cornea for ocular surface regeneration.

2. The composition of claim 1, further comprising a gelling agent selected from the group consisting of hydroxypropyl methylcellulose, hydroxypropyl cellulose, and methyl cellulose present in 1.0% to 15% (w/v).

3. The composition of claim 1, further comprising a poloxamer or carbomer.

4. The composition of claim 1, wherein the composition is an eyedrop formulation as a free-flowing liquid.

5. The composition of claim 1, wherein the composition is a gel as a viscous transparent liquid applied directly to the cornea of an eye.

6. The composition of claim 1, wherein the amniotic or chorionic collagen is obtained from a bovine placenta.

7. The composition of claim 1, wherein the collagen is obtained from a placental amnion wherein the chorion is removed.

8. The composition of claim 1, wherein the blend of type III and type IV collagen is crosslinked to control its degradation rate.

9. The composition of claim 1, wherein the blend of type III and type IV collagen is dispersed in a water-based solvent at room temperature to reach a concentration of 0.01% (w/v) to 5.0% (w/v), and one or more excipients selected from a gelling agent, a demulcent, and a viscosity enhancer is added to form a gel.

10. The composition of claim 1, wherein the blend of type III and type IV collagen in an isotonic solution is heated together at 37° C. with one or more excipients selected from the group consisting of a gelling agent, a demulcent, and a viscosity enhancer, to form a gel upon cooling.

11. The composition of claim 1, wherein the blend of type III and type IV collagen is blended with a thermoresponsive polymer at room temperature in an isotonic solution, to form a solution that forms a gel on application to the elevated temperature of the surface of an eye.

12. The composition of claim 11, wherein the thermoresponsive polymer is a poloxamer or carbomer.
